(19) United States
(12) Patent Application Publication
(10) Pub. No.: US 2004/0030390 A1
(43) Pub. Date: Feb. 12, 2004

(54) Intradiscal component installation apparatus and methods

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009, US

(21) Appl. No.: 10/421,433

(22) Filed: Apr. 23, 2003

Related U.S. Application Data
(60) Provisional application No. 60/374,747, filed on Apr. 23, 2002.

(51) Int. Cl. A61F 2/44

(52) U.S. Cl. 623/17.16; 623/17.15

(57) ABSTRACT

With respect to artificial disc replacements (ADRs) and total disc replacements (TDRs) having projections that penetrate vertebral endplates, methods and apparatus are disclosed that allow longer projections to be used for a given level of distraction. An inventive tool is used to install a second component in aligned registration subsequent to the installation of a first, opposing component. Inserting the components separately enables the use of longer projections. Although described in terms of “endplates,” the invention will work equally well in conjunction with intradiscal devices without a spacer; that is, with one or both components of a total disc replacement (TDR) that articulate directly against one another. In addition, although the disclosure illustrates the use of an instrument that fits into a cylinder-like concavity, compatibility with other geometric shapes and grasping features is clearly anticipated, whether formed into the components or extending therefrom.

### REFERENCE TO RELATED APPLICATION

This application claims priority from U.S. Provisional Patent Application Serial No. 60/374,747, filed Apr. 23, 2002, the entire content of which is incorporated herein by reference.

### FIELD OF THE INVENTION

This invention relates generally to artificial disc replacements (ADRs) and total disc replacements (TDRs) having projections that penetrate vertebral endplates and, in particular, to methods and apparatus that allow longer projections to be used for a given level of distraction.

### BACKGROUND OF THE INVENTION

Many spinal conditions, including degenerative disc disease, can be treated by spinal fusion or artificial disc replacement (ADR). ADR has several advantages over spinal fusion. The most important advantage of ADR is the preservation of spinal motion. Spinal fusion eliminates motion across the fused segments of the spine. Consequently, the discs adjacent to the fused level are subjected to increased stress. The increased stress increases the changes of future surgery to treat the degeneration of the discs adjacent to the fusion. However, motion through an ADR also-allows motion through the facet joints. Motion across arthritic facet joints could lead to pain following ADR. Some surgeons believe patients with degenerative disease and arthritis of the facet joints are not candidates for ADR.

Current ADR designs do not attempt to limit the pressure across the facet joints or facet joint motion. Indeed, prior art ADR generally do not restrict motion. For example, some ADR designs place bags of hydrogel into the disc space. Hydrogel bags do not limit motion in any direction. In fact, bags filled with hydrogels may not provide distraction across the disc space. ADR designs with metal plates and polyethylene spacers may restrict translation but they do not limit the other motions mentioned above. The articular surface of the poly spacer is generally convex in all directions. Some ADR designs limit motion translation by attaching the ADR halves at a hinge.

FIG. 1A is a lateral view of a prior-art artificial disc replacement (ADR). FIG. 1B is an anterior view of a prior-art ADR. FIG. 1C is a drawing which shows the prior-art ADR in flexion, and FIG. 1D is a drawing which shows the device in extension. Note that, due to impingement, left bending as permitted by the typical prior-art device, increases pressure on the left facet, whereas right bending increases pressure on the right facet. Rotation increases pressure on the right facet and the left facet, and vice versa.

The alignment of one Artificial Disc Replacement (ADR) endplate (EP) relative to the other ADR EP is critical to the function of articulating ADRs. Many ADRs rely on movement between a convexity on one ADR EP and a concavity on the other ADR EP. Alternatively, convex spacers are used between concavities on the ADR EPs. Improperly aligned ADR EPs risk excessive surface wear from incongruent opposing articulating surfaces. Furthermore, improper alignment will decrease ADR motion.

The endplates of prior art ADRs are inserted simultaneously to assure proper alignment. Most ADRs are held in the disc space by the fit of projections from the ADR EP into the vertebra above and below the ADR. Inserting ADR EPs simultaneously limits the length of these projections, however.

### SUMMARY OF THE INVENTION

This invention is directed to artificial disc replacements (ADRs) and total disc replacements (TDRs) having projections that penetrate vertebral endplates and, in the preferred embodiments, to methods and apparatus that allow longer projections to be used for a given level of distraction. Broadly, a tool is used to install a second component in aligned registration subsequent to the installation of a first, opposing component. Inserting the components separately enables the use of longer projections.

In a preferred embodiment, a first ADR endplate (EP) is press fit into a respective vertebral endplate, and an instrument according to the invention is used to align and install a second EP after the first. Although the invention is described in terms of “endplates,” the technique will work equally well in situations without a spacer; that is, with one or both components of a total disc replacement (TDR) that articulate directly against one another. In addition, although the disclosure illustrates the use of an instrument that fits into a cylinder-like concavity, compatibility with other geometric shapes and grasping features is clearly anticipated, whether formed into the components or extending therefrom.

### BRIEF DESCRIPTION OF THE DRAWINGS

FIG. 1A is a lateral view of a prior art artificial disc replacement (ADR);

FIG. 1B is an anterior view of a prior-art ADR;

FIG. 1C is a drawing which shows a prior-art ADR in flexion;

FIG. 1D is a drawing which shows the device in extension;

FIG. 2 is a simplified drawing of a restricted motion ADR according to the present invention;

FIG. 3A is a drawing of the embodiment of FIG. 2 in flexion;

FIG. 3B is a drawing of the embodiment of FIG. 2 in extension;

FIG. 3C is an anterior view of the embodiment of FIG. 2 attached to adjacent vertebrae;

FIG. 3D is a drawing of the embodiment of FIG. 2 illustrating how lateral bending is limited by contact on the left when bending is to the left, and on the right when bending is to the right;

FIG. 3E is a lateral view of a restricted motion ADR according to the invention;

FIG. 4 is a drawing of an alternative embodiment of the invention;

FIG. 5 is a side-view drawing which illustrates a way in which screws may be used to fix an ADR;

FIG. 6 is a drawing which shows the use of anterior flanges;

FIG. 7A is a side-view drawing of a further alternative embodiment according to the invention;

FIG. 7B shows the flange device of FIG. 7A in flexion;

FIG. 8 is a side-view drawing showing the use of an anterior check rein to prevent extension, for example;

FIG. 9 depicts the use of cross-coupled check reins;

FIG. 10 illustrates the optional use of an anterior flange configured to inhibit extension;

FIG. 11A is a drawing which illustrates yet a different embodiment of the invention;

FIG. 11B is a drawing which shows the device of FIG. 11A in flexion;

FIG. 11C shows the device in extension;

FIG. 11D is a side-view drawing of the way in which screws may be used to hold the device of FIG. 11D in place;

FIG. 11E an A-P view;

FIG. 12 is a side-view drawing which shows the area that could be removed to customize the vertebrae;

FIG. 13 is a first version according to this embodiment illustrating rotation surface(s);

FIG. 14 is a side-view drawing which shows a partial rotation surface received by a concavity in the imposing endplate;

FIG. 15A is an end-view of an ADR according to the invention placed on the vertebrae seen from a top-down A-P view;

FIG. 15B is a drawing of the embodiment of FIG. 15A with the ADR and axle rotated;

FIG. 16 is a drawing which shows a removable alignment guide used for placement of this embodiment;

FIG. 17 is a simplified cross-sectional view of a patient on an operating table, showing the alignment guide in position;

FIG. 18A is a lateral view using fluoroscopy which shows the circular cross-section of the axle when properly aligned;

FIG. 18B is an anterior view of this alternative embodiment;

FIG. 18C is an anterior view;

FIG. 19A shows how disc space is distracted;

FIG. 19B shows the impact distraction element in place between the end plates;

FIG. 19C shows the tool being manipulated to spread the vertebrae apart;

FIG. 19D shows a third step how the end plates are prepared through the use of a reamer and/or circular grinder;

FIG. 19E shows a first end plate for the final ADR is inserted;

FIG. 19F shows how the second end plate is inserted;

FIG. 19G show how the end plates are optionally screwed into place;

FIG. 19H shows the step of inserting an axle between the end plates;

FIG. 19I shows the anterior poly block snapped in position on the other side of the installed axle;

FIG. 20 is an anterior view of the ADR installed between opposing vertebrae;

FIG. 21 shows the use of optional wedges or convex pieces to attach the ADR end plate;

FIG. 22 is a drawing which shows an inventive cutting guide having a curved end to prevent saw from cutting into the nerves;

FIG. 23A is a side-view drawing of a further, different embodiment of the invention utilizing a hinged axle; and

FIG. 23B is an end view of the embodiment of FIG. 23A shown without flexion/extension blocks to better illustrate the hinged portion.

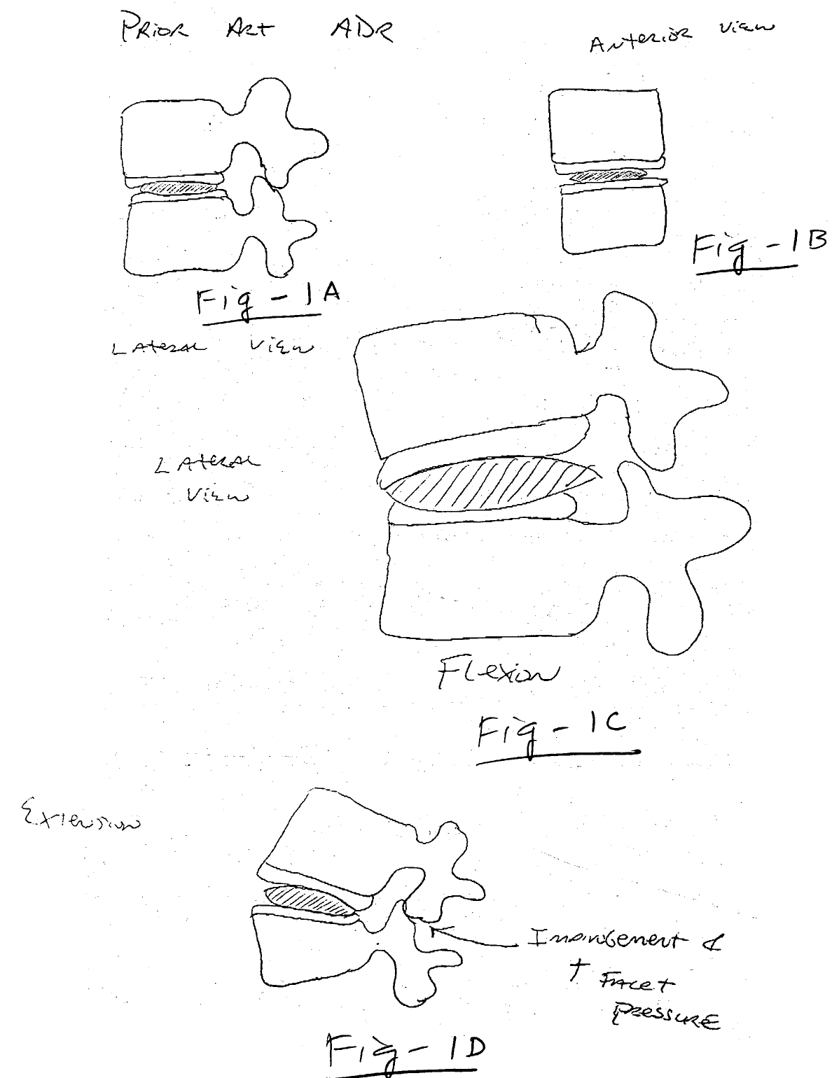

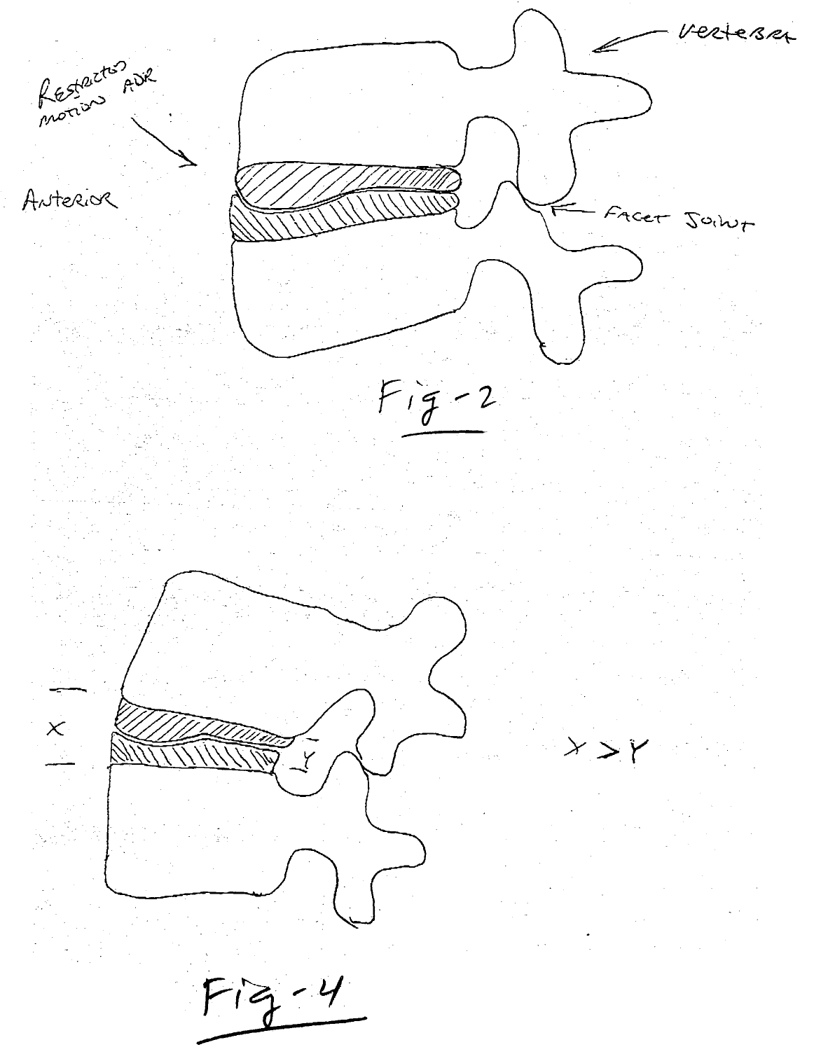

### DETAILED DESCRIPTION OF THE INVENTION

The present invention limits both facet joint pressure and facet joint motion. Broadly, the pressure on the facet joints is lowered from the preoperative pressure by distracting the disc space. The present invention also reduces the facet joint pressure by eliminating or significantly reducing motion across the ADR that increase the pressure on the facet joints. Specifically, ADR design in accordance with the various embodiments restricts spinal extension, rotation, translation, and lateral bending. Forward flexion is not restricted as forward flexion decreases the pressure on the facet joints.

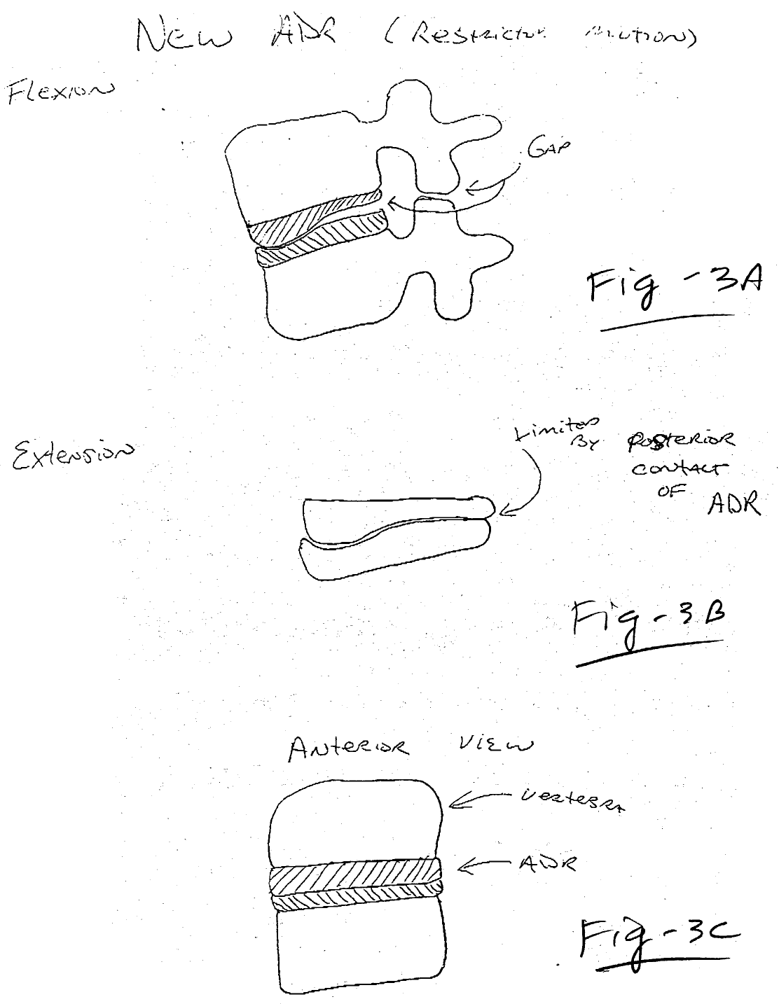

FIG. 2 is a simplified drawing of a restricted motion artificial disc replacement (ADR) according to this invention. FIG. 3A is a drawing of the embodiment of FIG. 2 in flexion, illustrating the way in which gaps are created in the posterior of the vertebrae and the facet joint. FIG. 3B is a drawing of the embodiment of FIG. 2 in extension, showing how posterior contact is limited. FIG. 3C is an anterior view of the embodiment of FIG. 2 attached to adjacent vertebrae. FIG. 3D is a drawing of the embodiment of FIG. 2 illustrating how lateral bending is limited by contact on the left when bending is to the left, and on the right when bending is to the right. FIG. 3E is a lateral view of a restricted motion ADR according to the invention, illustrating how rotation and translocation are limited by a spoon-on-spoon cooperation.

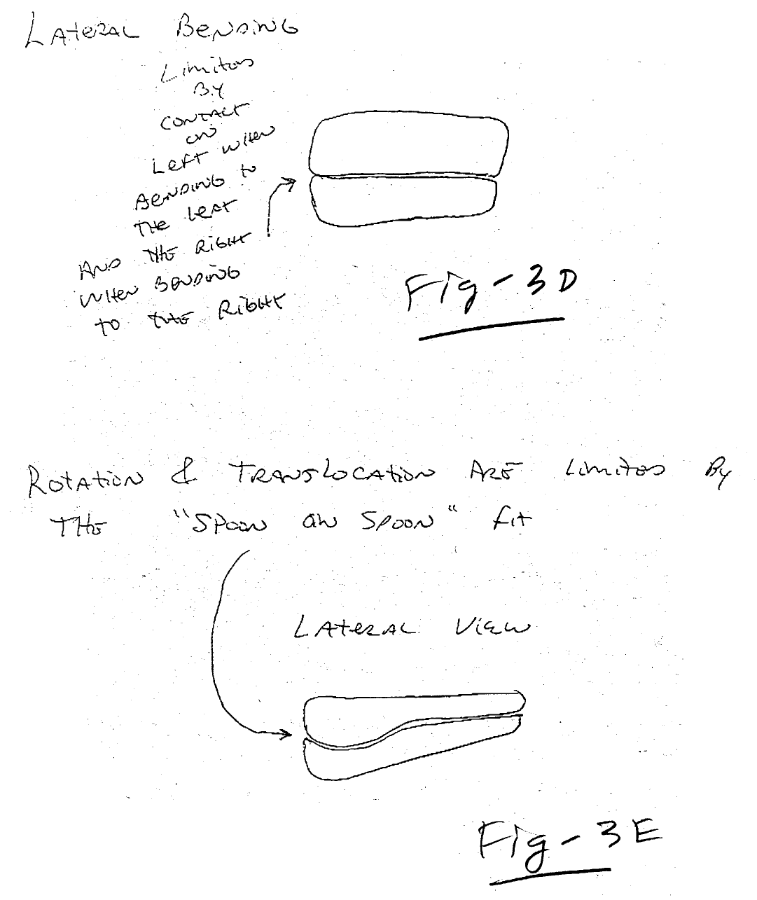

FIG. 4 is a drawing of an alternative embodiment of the invention, illustrating how a wedge or trapezoid-shaped ADR may be used according to the invention to preserve lordosis. FIG. 5 is a side-view drawing which illustrates a way in which screws may be used to fix an ADR according to the invention to upper and lower vertebrae. In particular, a fastener may be used having coarse threads received by the bone, and finer threads associated with actually locking the ADR into place. FIG. 6 is a drawing which shows the use of anterior flanges facilitating the use of generally transverse as opposed to diagonally oriented screws.

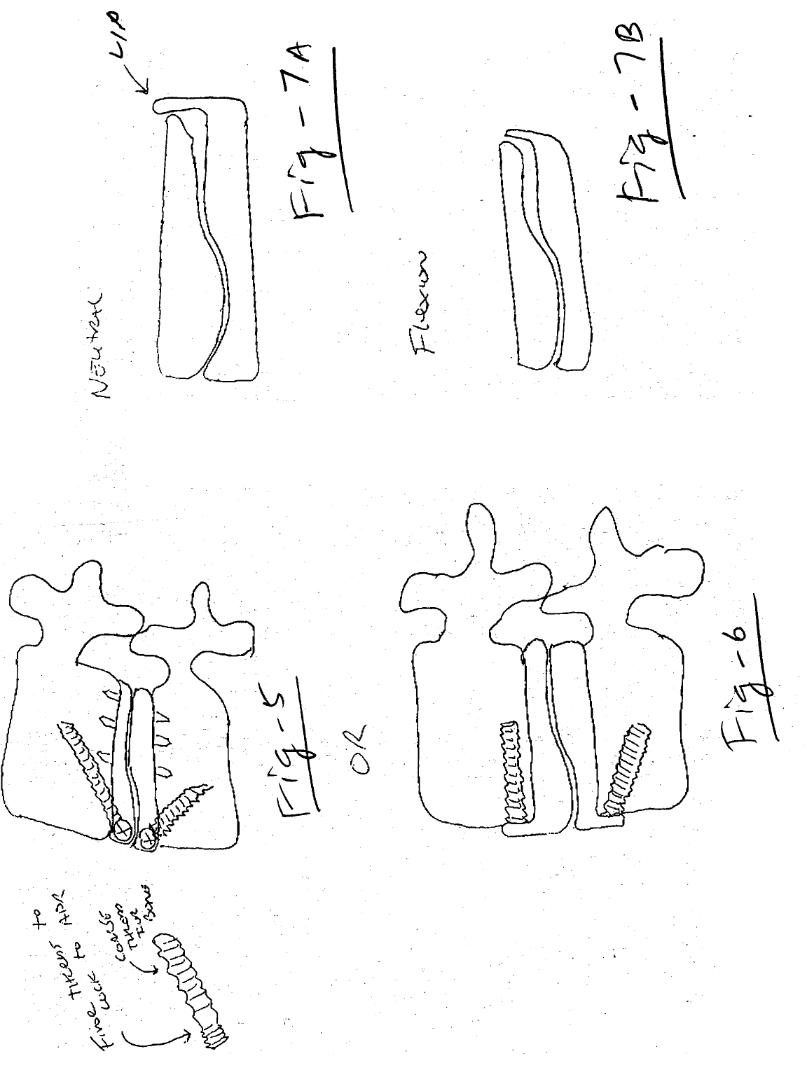

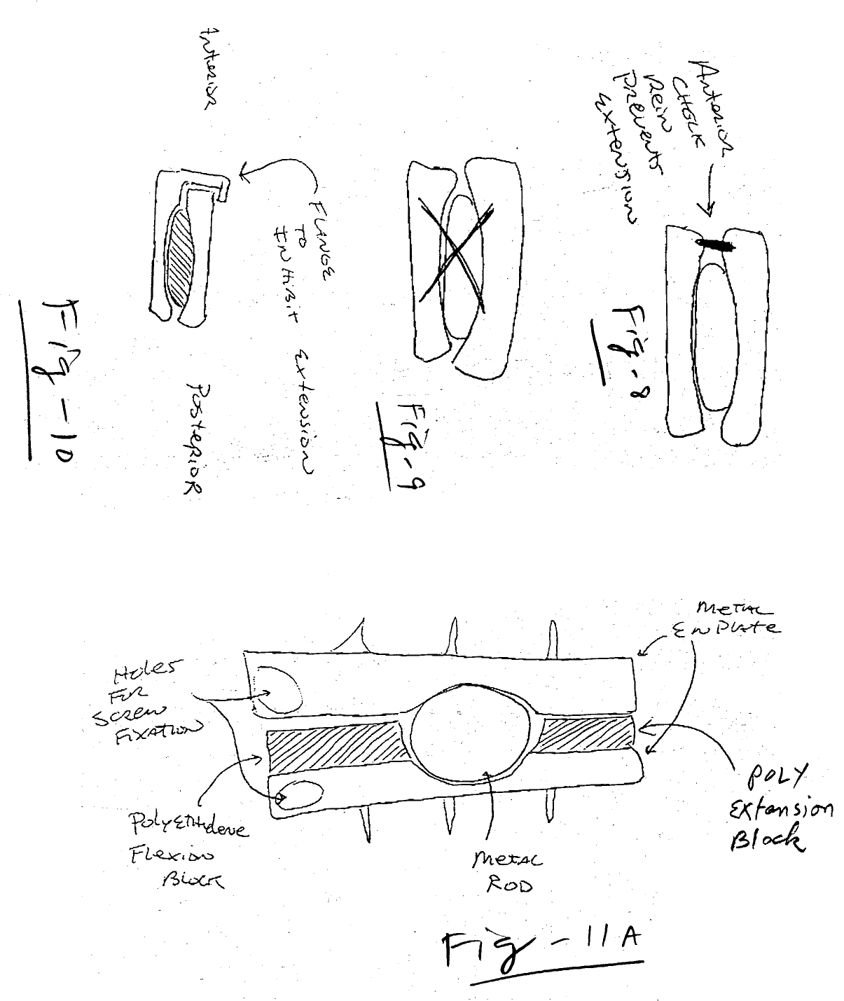

FIG. 7A is a side-view drawing of a further alternative embodiment according to the invention, featuring an optional lip to prevent the trapping of soft tissue during the movement from a flexion to neutral position. FIG. 7B shows the flange device of FIG. 7A in flexion. As a substitute for, or in conjunction with, peripheral flanges, check reins may be used to restrict motion. FIG. 8 is a side-view drawing showing the use of an anterior check rein to prevent extension, for example. Lateral check reins may be used to prevent lateral bending, and cross-coupled check reins may be used to prevent translation. FIG. 9 depicts the use of cross-coupled check reins. FIG. 10 illustrates the optional use of an anterior flange configured to inhibit extension.

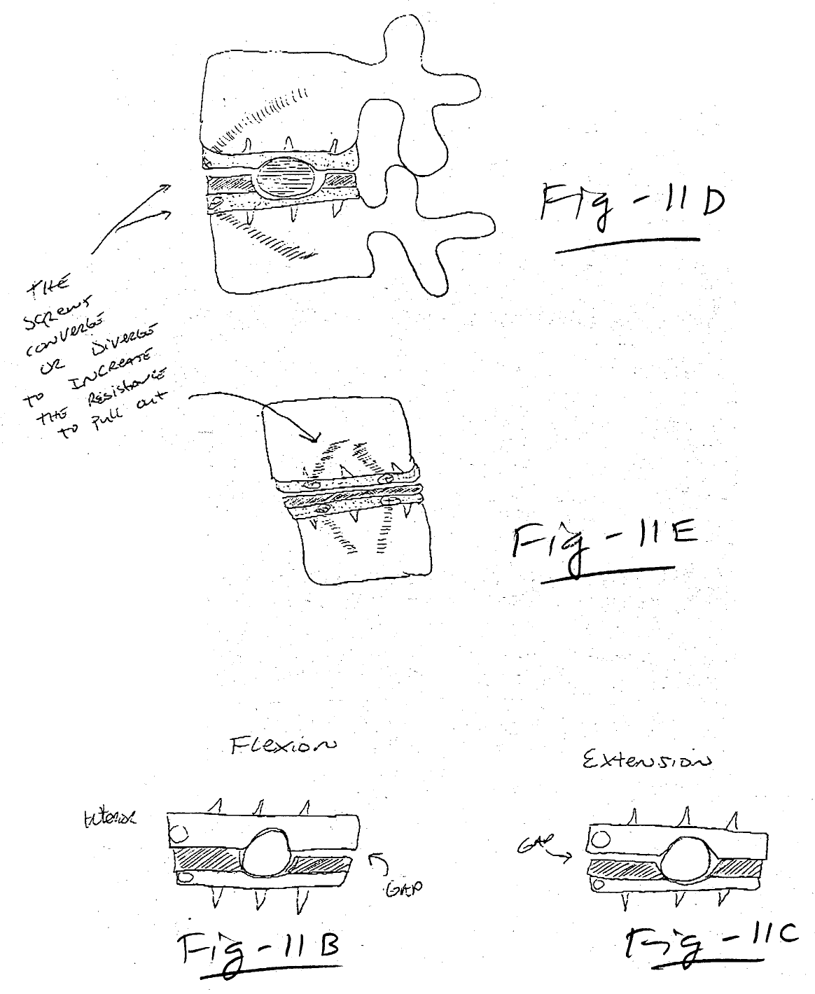

FIG. 11A is a drawing which illustrates yet a different embodiment of the invention, including the use of flexion and/or extension blocks. Shown in the figure, endplates, preferably metal, include recesses to receive a centralized rod, also preferably metallic. On either side of the rod, but between the end plates, there is disposed a more wearing bearing block of material such as polyethylene, one preferably associated with flexion and an opposing block associated with extension. Holes may be provided for fixation along with projections for enhanced adherence. FIG. 11B is a drawing which shows the device of FIG. 11A in flexion, and FIG. 11C shows the device in extension. Note that, during flexion, a posterior gap is created, whereas, in extension, an anterior gap is created. In this embodiment, the degree of flexion and extension may be determined by the thickness of the flexion/extension blocks, which may determined at the time of surgery. FIG. 11D is a side-view drawing of the way in which screws may be used to hold the device of FIG. 11D in place. FIG. 11E an A-P view. Note that the screws may converge or diverge, to increase resistance to pull-out.

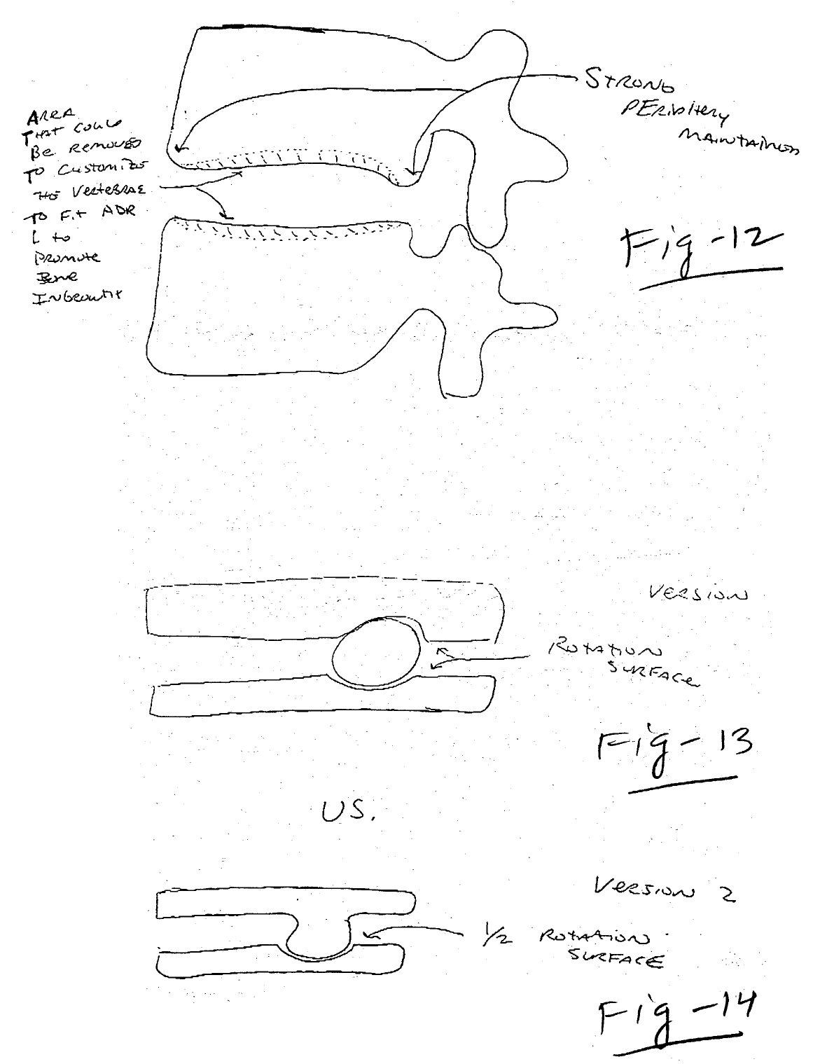

The superior surface of the superior endplate and the inferior surface of the inferior endplate of the ADR could be convex. The convex surfaces of the ADR would fit the concavities of the endplates of the vertebrae. The endplates could be decorticated to promote bone ingrowth into the endplates of the ADR. An expandable reamer or a convex reamer could preserve or increase the concavities. The concavities have two important advantages. First, they help prevent migration of the ADR. The convexities of the ADR fit into the concavities of the vertebrae. Second, the majority of support for the ADR occurs at the periphery of the vertebral endplates. Thus, reaming away a portion of the central, concave, portion of the vertebrae promotes bone ingrowth through exposure to the cancellous interior of the vertebrae, yet preserves the stronger periphery. FIG. 12 is a side-view drawing which shows the area that could be removed to customize the vertebrae so as to fit an ADR according to the invention and/or promote ingrowth.

The endplates of the ADR could be any material that promotes bone ingrowth. For example, titanium or chrome-cobalt with a porous, beaded, or plasma spray surface. The flexion and extension blocks would likely be made of polyethylene, but could also be made of other polymers, ceramic, or metal. The central rod or axle would likely made of the same metal as the endplates of the ADR, but could also be made of polyethylene or other polymer, or ceramic. A metal or ceramic rod would have better surface wear than a polyethylene rod. A limited amount of compression to axial loads could occur when a portion of the ADR endplates lie against the polyethylene blocks. A central rod is preferred over incorporating a raised rod like projection into one of the endplates. The central rod allows rotation about twice as much surface area (the superior and inferior surfaces). The increased surface area decreases the pressure on the surface during rotation about the central axle/rod. FIG. 13 is a first version according to this embodiment illustrating rotation surface(s). FIG. 14 is a side-view drawing which shows a partial rotation surface received by a concavity in the imposing endplate. Both versions shown in FIGS. 13 and 14 are assembled within the disc space.

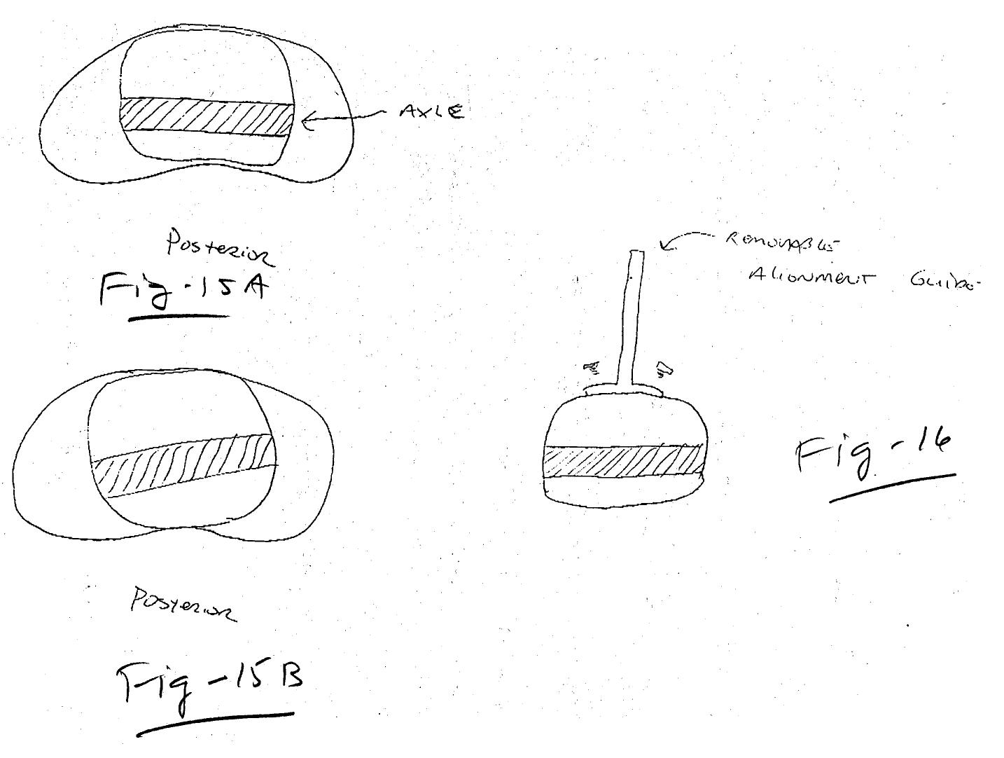

Alignment of the ADR is critical. If the central rod or axle is diagonal to the long axis of the vertebral endplate, the patient will bend to the left or right while bending forward. Novel (for and ADR) alignment guides are described below. Furthermore, if the axle is made of polyethylene, metallic markers will be incorporated into the ends of the axle. Surgeons can assure proper alignment by fluoroscopic images during surgery. FIG. 15A is a end-view of an ADR according to the invention placed on the vertebrae seen from a top-down A-P view. FIG. 15B is a drawing of the embodiment of FIG. 15A with the ADR and axle rotated. Should the patient have trouble bending forward, and so forth, the patient may twist at the side while bending forward, as appropriate.

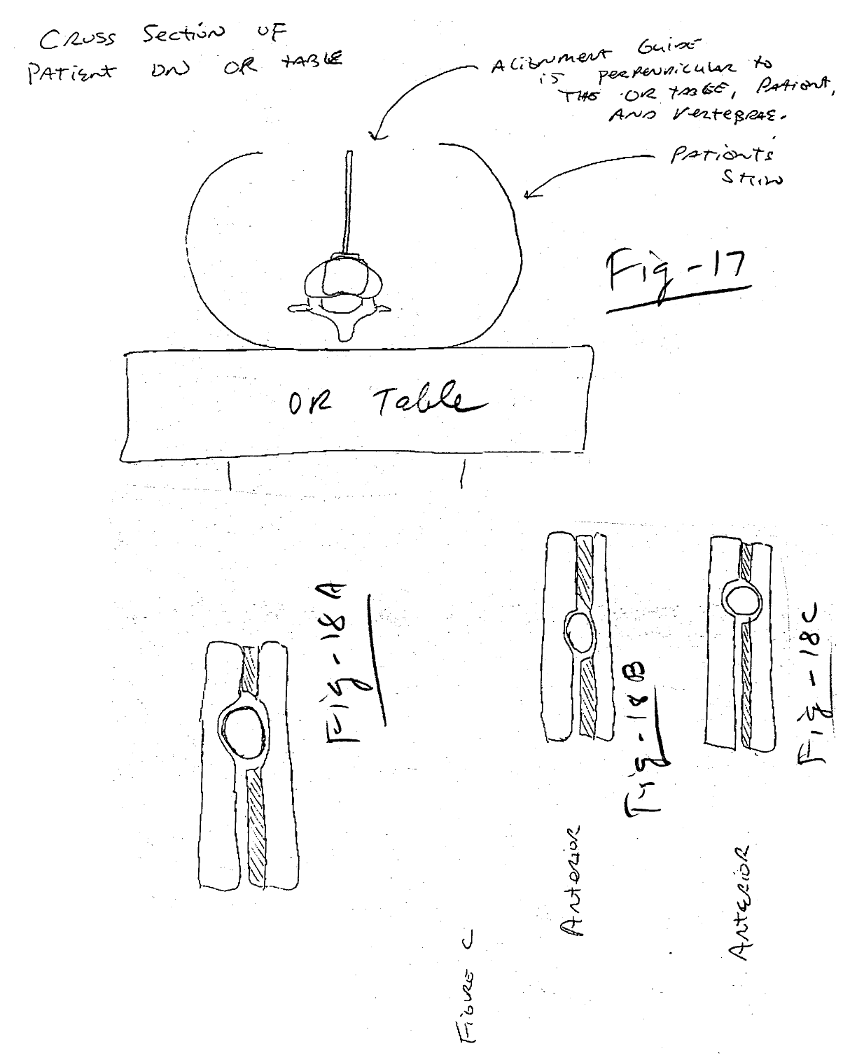

FIG. 16 is a drawing which shows a removable alignment guide used for placement of this embodiment. FIG. 17 is a simplified cross-sectional view of a patient on an operating table, showing the alignment guide in position. In particular, the alignment guide is preferably perpendicular to the table, the patient, and vertebrae with respect to al proper orientation. FIG. 18A is a lateral view using fluoroscopy which shows the circular cross-section of the axle when properly aligned.

The ADR endplates could be designed to locate the axle transversely in any location from anterior to posterior. The location may vary depending on the disc that will be replaced. For example, the axle may located at the junction of the anterior ⅔^{rd} and posterior ⅓^{rd} for the L5/S1 disc but at the anterior 1/2 and posterior 1/2 for the L3/L4 disc. Similarly, the degree of wedge shape will vary with the disc to be replaced. L5/S1 will require a more wedge shaped ADR than L3/L4. FIG. 18B is an anterior view of this alternative embodiment, and FIG. 18C is an anterior view.

Preoperative templates will be provided to help the surgeon predict which ADR will be needed. The ADR could be inserted fully assembled or constructed in the disc space. Construction within the disc space allows the surgeon to force spikes of the ADR endplate into the vertebrae. Assembly in the disc space also allows maximum use of the vertebral concavities. The polyethylene blocks contain features to allow them to snap into place. Polyethylene trays with “snap” features are well described in the total knee replacement literature.

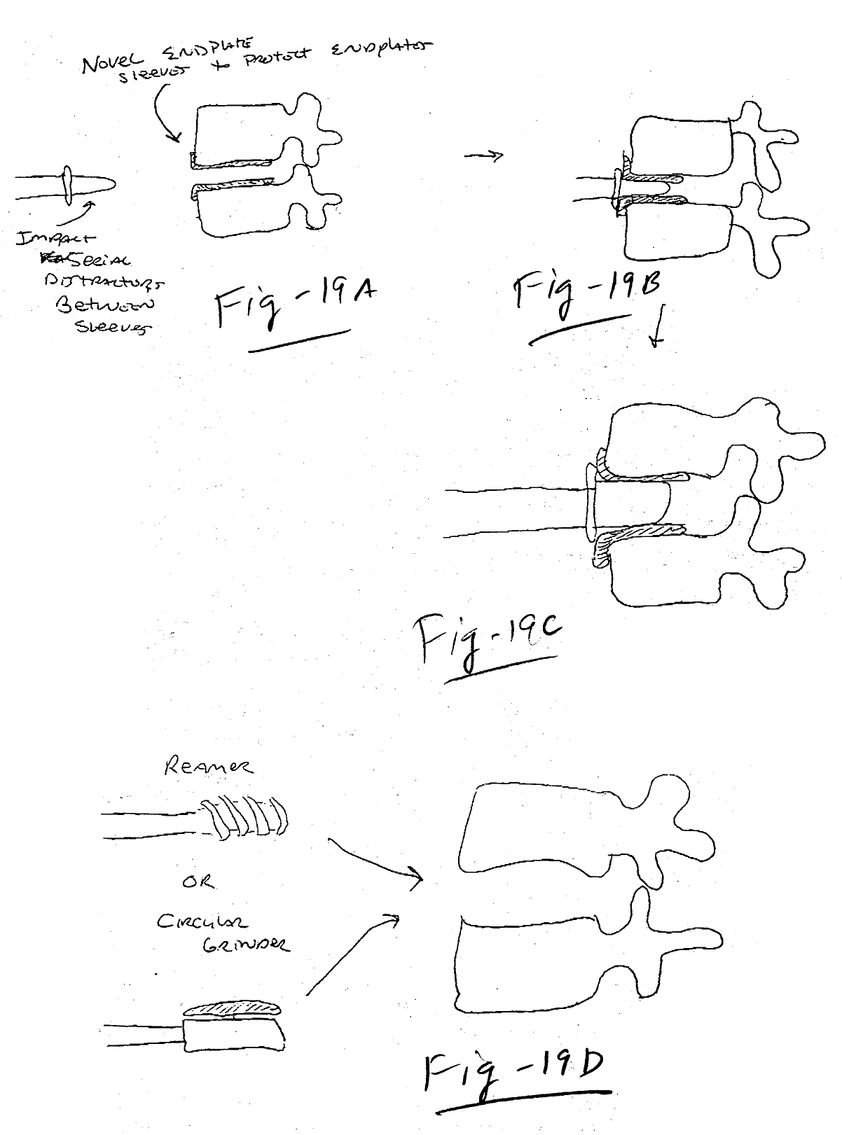

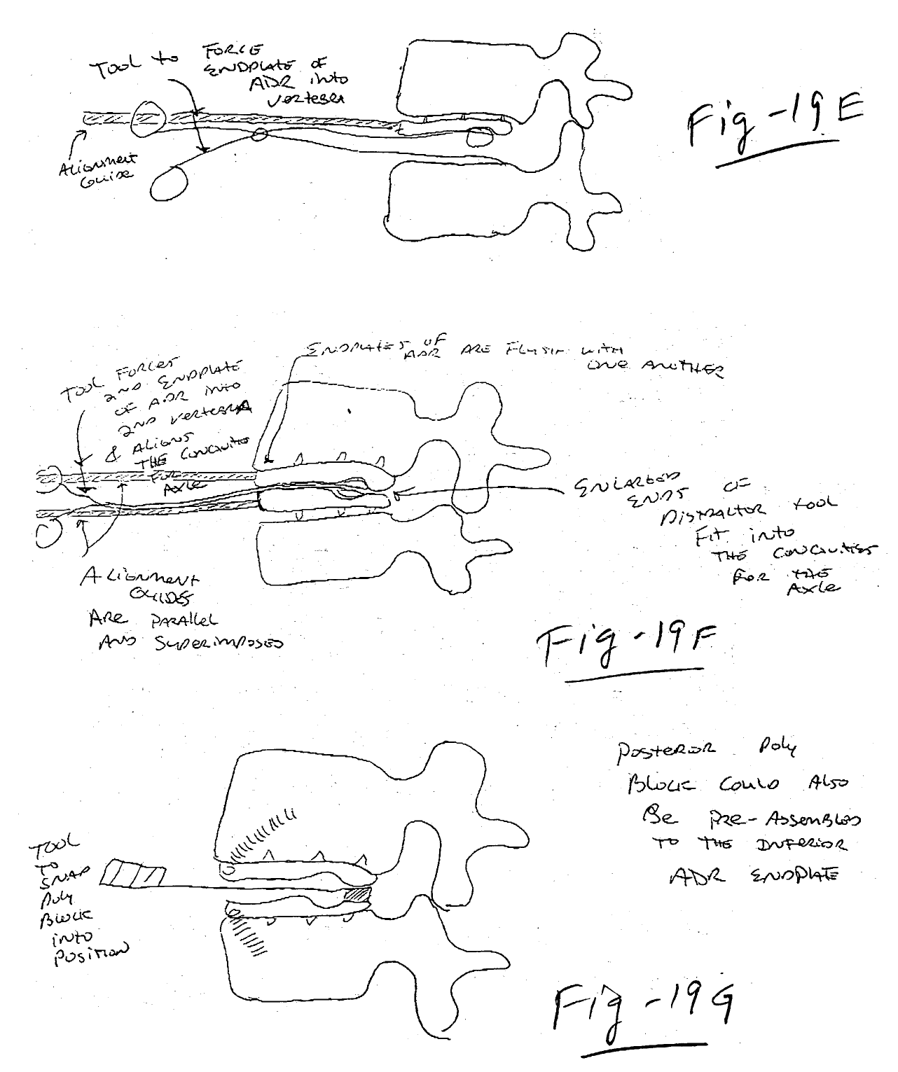

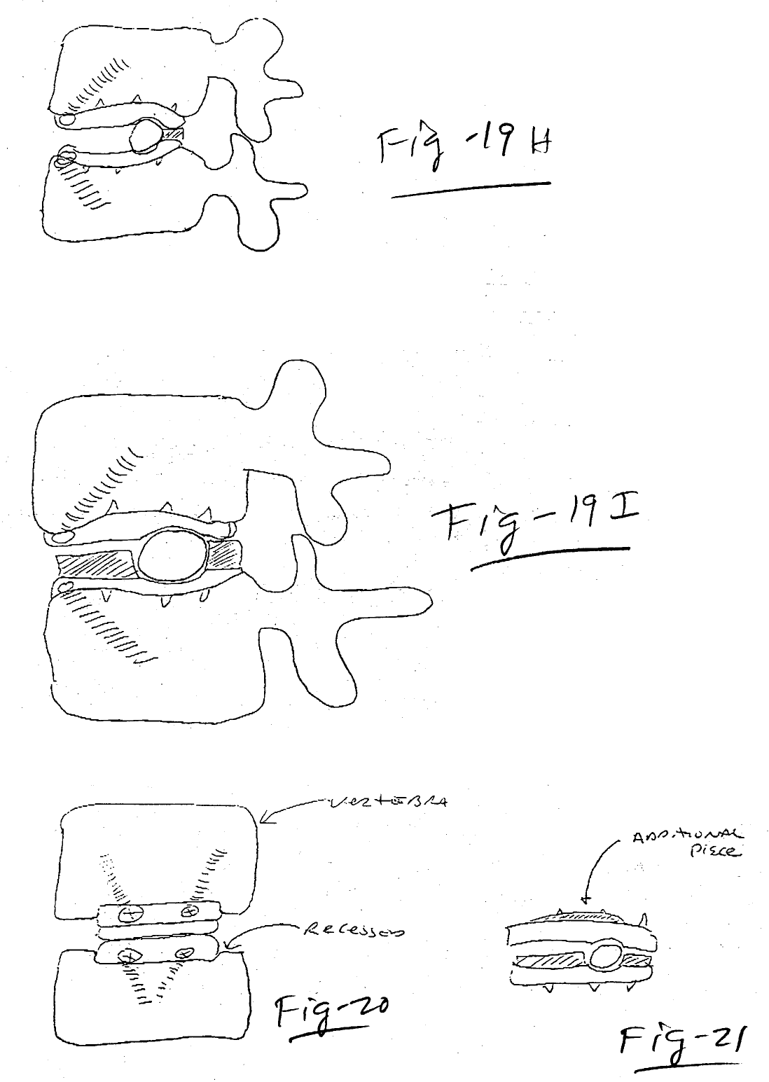

FIGS. 19A-19I illustrate steps associated with installing a restricted motion ADR according to the invention. In the preferred embodiment the ADR relies on bone ingrowth. Alternatively, the ADR may be cemented to the vertebrae using, for example, methyl methacrylate. Novel, safer cutting guides, and a novel distraction instruments are described. The system also provides trial implants and instruments to determine the balance and tension of the surrounding soft tissues.

As an initial step, a portion of the disc annulus and most or all of the disc nucleus are removed (not shown). As a second step, the disc space is distracted, as shown in FIG. 19A. In this case a novel implant sleeve is used to protect the end plates, and an impact serial distracter is used between these sleeves. FIG. 19B shows the impact distraction element in place between the end plates, and FIG. 19C shows the tool being manipulated to spread the vertebrae apart.

According to a third step, the end plates are prepared through the use of a reamer and/or circular grinder with the distraction sleeves removed, as shown in FIG. 19D. As a fourth step, the trial ADR is inserted (not shown) so as to select a proper size ADR (step 5, also not shown). Having determined the proper size, a first end plate for the final ADR is inserted as shown in FIG. 19E with a tool used to force the end plate of the ADR into the vertebrae, whether upper or lower.

This section of the disclosure emphasizes methods and instruments that allow for the separate insertion of ADR EPs. Aligning the insertion of a second ADR EP relative to a first EP that enables the use of longer projections from the ADR EPs, resulting in a more controlled procedure. Referring to FIGS. 19E and 19F in particular, the upper ADR EP has been press fit into the vertebra above the disc space. A special tool fits into a portion of the ADR EP that was inserted first, thereby aligning the insertion of the second ADR. The tool can also be used to press the second ADR EP into the vertebra. Although FIGS. 19E and 19F illustrate the use of an instrument that fits into cylinder-like concavities, the instrument could fit into other shapes in the ADR EPs, including slots and other shapes with flat sides.

In FIG. 19F, the second end plate is inserted, such that the opposing end plates are flush with one another. The tool used for this purpose forces the second plate of the ADR into the second vertebrae while simultaneously aligning the concavities to receive the axle. Alignment guides may be used in parallel/superimposed fashion to ensure that the opposing end plates are oriented properly. In addition, the enlarged ends of the distraction tool may include end features which fit into the cavities for axle, again, to ensure proper orientation. In step 8, shown in FIG. 19G, the end plates are optionally screwed into place, and a first poly block is installed posteriorly using a tool to snap the block into position. Note that the posterior poly block may also be preassembled to the inferior ADR end plate, as an option.

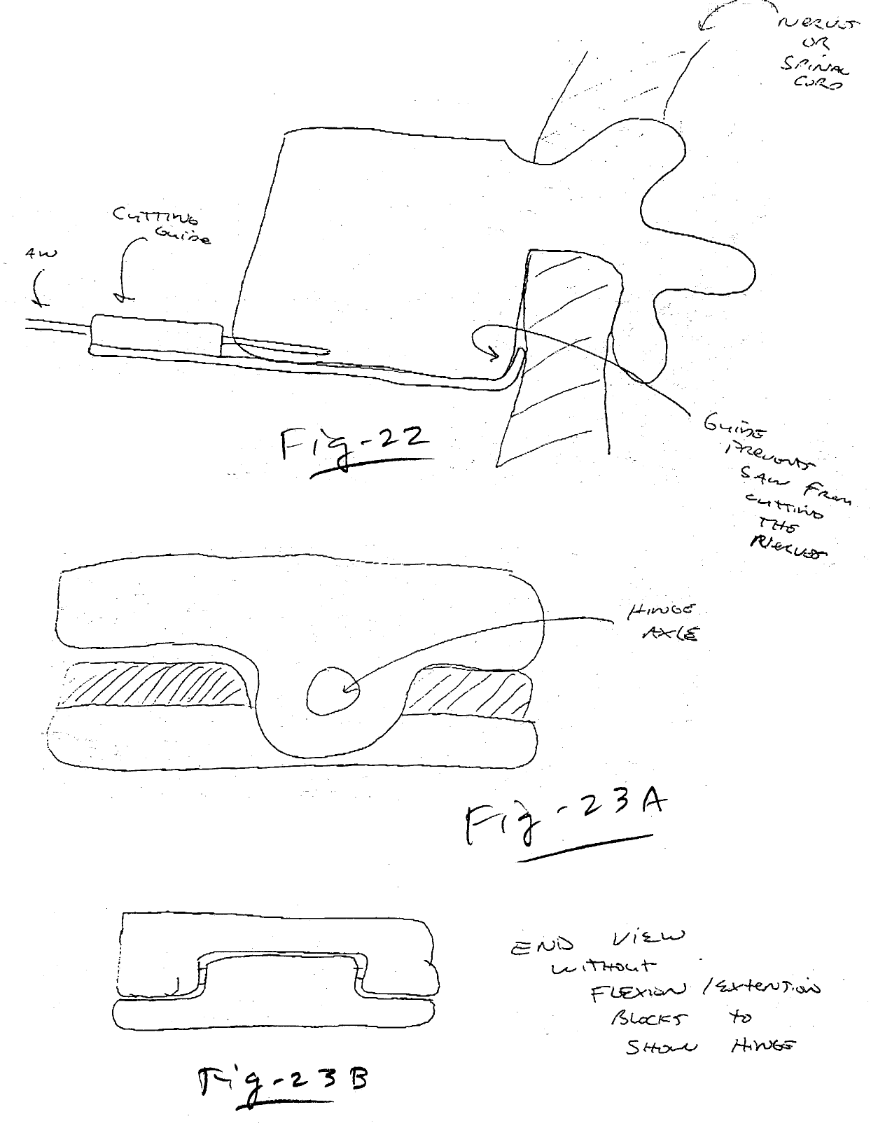

FIG. 19H shows the step of inserting an axle between the end plates. In step 10, shown in FIG. 19I, the anterior poly block is snapped in position on the other side of the installed axle. The ADR could be placed into recessed areas of the vertebrae to help hold it in place. FIG. 20 is an anterior view of the ADR installed between opposing vertebrae also showing the relative positioning of recesses formed in the end plates of the vertebrae. FIG. 21 shows the use of optional wedges or convex pieces to attach the ADR end plate so as to customize the prosthesis to a particular patient anatomy.

FIG. 22 is a drawing which shows an inventive cutting guide having a curved end to prevent saw from cutting into the nerves. FIG. 23A is a side-view drawing of a further, different embodiment of the invention utilizing a hinged axle. FIG. 23B is an end view of the embodiment of FIG. 23A shown without flexion/extension blocks to better illustrate the hinged portion.

I claim:

1. Intradiscal apparatus for use between upper and lower vertebral bodies with opposing endplates, the system comprising:
a superior component configured for installation against the endplate of the upper vertebral body;
an inferior component configured for installation against the endplate of the lower vertebral body;
each component including a grasping feature that is physically aligned with the grasping feature of the other component; and
an instrument physically couplable to the grasping features, enabling one of the components to be installed in alignment with the other component after the other component has been installed.

2. The apparatus of claim 1, wherein the components are prosthetic endplates associated with an artificial disc replacement (ADR).

3. The apparatus of claim 1, wherein one or both of the components include one or more projections adapted to penetrate a respective vertebral endplate.

4. The apparatus of claim 1, wherein:
one or both of the components include one or more projections adapted to penetrate a respective vertebral endplate; and
the ability to install the components in sequence allows longer projections to be used for a given level of distraction.

5. The apparatus of claim 1, wherein the grasping features are concavities formed in the components.

6. The apparatus of claim 5, wherein the concavities are cylindrical tunnels.

7. A method of implanting a disc replacement, comprising the steps of:
providing the apparatus of claim 1;
installing one of the components by compressing that component against a respective one of the vertebral endplates;
coupling the other component to the instrument; and
Installing the other one of the components against the other vertebral endplate by coupling the instrument to the other component.
